(19) United States
(12) Patent Application Publication
(10) Pub. No.: US 2019/0167932 A1
(43) Pub. Date: Jun. 6, 2019

(54) ORAL CARE SYSTEM METHOD AND KIT

(71) Applicant: AIRWAY MEDIX S.A., Warszawa (PL)

(72) Inventors: Oron ZACHAR, Tel Aviv (IL); Elad EINAV, Salit (IL)

(21) Appl. No.: 16/139,018

(22) Filed: Sep. 22, 2018

Related U.S. Application Data
(63) Continuation of application No. 14/383,940, filed on Sep. 9, 2014, now abandoned.
(60) Provisional application No. 61/609,331, filed on Mar. 11, 2012.

(51) Int. Cl. A61M 16/04; A61C 17/06; A61M 11/00

(52) U.S. Cl. CPC A61M 16/0409; A61C 17/04; A61M 16/0434; A61M 16/0445; A61M 16/0463; A61M 16/0459; A61M 2209/10; A61M 11/00; A61M 16/0484; A61M 2210/065; A61M 2205/0216; A61M 2210/0656; A61M 16/0486

(57) ABSTRACT

A system for oral care of patients ventilated with an ETT tube 120 comprising: a deformable fluid blocking element 200 reversibly mountable around an ETT (for example, side-mountable thereon), the fluid blocking element having: a midsection channel 205 sized to fit snugly and reversibly around the ventilation tube 120 when the fluid blocking element is mounted thereto; and an outer surface configured, when the fluid blocking element is mounted, to fit snugly and reversibly into the back of the mouth of an adult human patient so as to span both lateral left-to-right tonsils and tongue-soft palate dimensions thereof, wherein the fluid blocking element 200 is reversibly inflatable to simultaneously press against the soft pallet and the tongue and/or the fluid blocking element 200 is collapsible and outwardly biased so that when placed in the back of the human mouth, the fluid blocking element expands so as to simultaneously press against the soft pallet and the tongue. Methods and kits are also disclosed herein.

### CROSS-REFERENCE TO RELATED APPLICATIONS

The present application claims priority to U.S. 61/609,331 filed on Mar. 11, 2012 which is hereby incorporated by reference in its entirety.

### BACKGROUND AND RELATED ART

Endotracheal tubes (ETT) are known in the art—one non-limiting example (copied from US published patent application 20120097168 incorporated herein by reference) is illustrated in FIG. 1A. The ETT of FIG. 1A includes an expandable cuff for blocking fluid penetration into the trachea. The expandable cuff is permanently attached to a distal half of the ETT. Although the expandable cuff of FIG. 1A is inflatable—this is not a limitation. For example, WO/2010/151713 and WO/2012/176112 illustrate cuffs that do not necessarily rely on inflation. The endotracheal tube also includes a “Murphey eye” at a distal end of the ETT.

FIG. 1B illustrates various elements of the human oral cavity including: tongue 710, left 115 and right 114 tonsils, soft palate 740, uvula 112, and back teeth 110.

FIG. 1C refers to the situation where the human subject is intubated with an endotracheal tube (ETT) or a tracheostomy tube.

The following issued patents and patent publications provide potentially relevant background material, and are all incorporated by reference in their entirety: U.S. Pat. No. 7,866,477 entitled “Oral care Q2 kits”; U.S. Pat. No. 4,984,984 entitled “Dental tool and nozzle therefor”; and WO 2005/070324 entitled “Droplet Jet System For Cleaning”, WO 1995/006492 entitled “Cuffed Oro-Pharyngeal Airway and U.S. Pat. No. 2,099,127 entitled ““Pharyngeal Bulb Gasway.”

### SUMMARY OF EMBODIMENTS

It is now disclosed a reversibly/side mountable system for oral care of patients ventilated with an endotracheal tube (ETT), the system comprising: a deformable fluid blocking element reversibly mountable and side-mountable around the ETT, the fluid blocking element having: (i) a midsection channel sized to fit snugly and reversibly around the ETT when the fluid blocking element is mounted thereto; and (ii) an outer surface configured, when the fluid blocking element is mounted, to fit snugly and reversibly into the back of the mouth of an adult human patient so as to span both lateral left-to-right tonsils and tongue-soft palate dimensions thereof, wherein the fluid blocking element is configured so that when mounted to the ETT, (A) the fluid blocking element is reversibly inflatable to simultaneously press against the soft palate and the tongue and/or (B) the fluid blocking element is collapsible and outwardly biased so that when placed in the back of the human mouth, the fluid blocking element expands so as to simultaneously press against the soft palate and the tongue.

The term ‘reversibly mountable’ is as opposed to ‘permanently attached.’ The term ‘side-mountable’ refers to ‘at least’ side-mountable. When a fluid-blocking element is both ‘reversibly mountable’ and ‘side-mountable’ to an ETT of an intubated and ventilated patient, it is possible, during a time of ventilation via the ETT, (i) to mount the fluid blocking element to the ETT and (ii) to subsequently remove the fluid blocking therefrom with no need to even briefly interrupt ventilation of the patient. While the fluid blocking element is mounted, the patient may be continuously ventilated—for example, while his/her teeth are cleaned—e.g. so that the fluid blocking element substantially blocks cleaning fluid and/or debris within the oral cavity from penetrating into an airway location below the epiglottis of the human subject of the patient.

In some embodiments, the deformable fluid blocking element is configured so that when mounted to the ETT, the fluid blocking element does not fully surround the ETT.

In some embodiments, the deformable fluid blocking element does not contain a permanent ring.

In some embodiments, the deformable fluid blocking element is configured so that when mounted to the ETT, the fluid blocking element does not fully surround the ETT with a permanent ring.

In some embodiments, the deformable fluid blocking element is configured so that when mounted to the ETT, the fluid blocking element fully surrounds the ETT with a reversibly openable ring.

It is now disclosed a kit for oral care of patients ventilated with an endotracheal tube (ETT), the kit comprising: a. at least one spray device configured to deliver a spray; and b. a deformable fluid blocking element reversibly mountable around the ETT, the fluid blocking element having:

i. a midsection channel sized to fit snugly and reversibly around the ETT when the fluid blocking element is mounted thereto; and ii. an outer surface configured, when the fluid blocking element is mounted, to fit snugly and reversibly into the back of the mouth of an adult human patient so as to span both lateral left-to-right tonsils and tongue-soft palate dimensions thereof, wherein the fluid blocking element is configured so that when mounted to the ETT, (A) the fluid blocking element is reversibly inflatable to simultaneously press against the soft palate and the tongue and/or (B) the fluid blocking element is collapsible and outwardly biased so that when placed in the back of the human mouth, the fluid blocking element expands so as to simultaneously press against the soft palate and the tongue.

In some embodiments, spray device is a hybrid spray-suction device configured to both deliver the spray and to suction away material (i.e. liquid and/or solid material).

In some embodiments, the spray device is a hybrid spray-suction device configured to simultaneously deliver the spray and to suction away material.

In some embodiments, the deformable fluid blocking element is reversibly mountable and side-mountable around the ETT.

In some embodiments, the spray device includes first and second inlet respectively configured to receive a liquid stream and a gas stream and is configured to form the spray from the received liquid and gas streams.

It is now disclosed a entilation system comprising: a. an endotracheal tube (ETT); and b. a deformable fluid blocking element reversibly mounted around the ETT, the fluid blocking element having: a midsection channel sized to fit snugly and reversibly around the ETT when the fluid blocking element is mounted thereto; and an outer surface configured, when the fluid blocking element is mounted, to fit snugly and reversibly into the back of the mouth of an adult human patient so as to span both lateral left-to-right tonsils and tongue-soft palate dimensions thereof, wherein (i) the fluid blocking element is reversibly inflatable to simultaneously press against the soft palate and the tongue and/or (ii) the fluid blocking element is collapsible and outwardly biased so that when placed in the back of the human mouth, the fluid blocking element expands so as to simultaneously press against the soft palate and the tongue.

In some embodiments, the fluid blocking element is reversibly mounted around a proximal half of the ETT.

In some embodiments, further comprising: c. a fluid-blocking cuff and/or inflatable balloon permanently mounted to the ETT at a location distal to the fluid blocking element.

In some embodiments, the fluid-blocking cuff is configured so that when the ETT is deployed within the trachea to define an interstitial region outside of the ETT and within the trachea, the fluid-blocking cuff, is expandable to substantially seal an upper portion of the interstitial region from a lower portion thereof.

It is now disclosed an oral cleaning method comprising: a. inserting a deformable fluid blocking element into a mouth of an adult human subject who is ventilated with an endotracheal tube (ETT); the fluid blocking element having a midsection channel sized to fit snugly and reversibly around the ETT, the fluid blocking element being inserted so the ETT passes through the midsection channel; b. concurrent with or subsequent to the insertion, expanding the deformable fluid blocking element so that an outer surface thereof fits snugly and reversibly into the back of the mouth of the adult human subject so as to span both lateral and tongue-soft palate dimensions thereof; and c. introducing (e.g. spraying or otherwise forcing) a cleaning fluid into the mouth of human subject so as to clean teeth thereof, wherein the fluid blocking element substantially blocks the cleaning fluid from penetrating into an airway of the human subject.

The introduced fluid may be a liquid or a liquid-gas mixture such as a spray or mist.

In some embodiments, the fluid is introduced as a spray and/or the fluid comprises a gas-liquid mixture.

In some embodiments, a pressurized fluid is introduced.

In some embodiments, further comprising suctioning away cleaning fluid which is retained in the mouth by the fluid blocking element in locations proximal thereto.

In some embodiments, at least some of the suctioned cleaning fluid is suctioned out of the mouth via enters via a suction port deployed substantially on an upper surface of the fluid blocking element.

In some embodiments, the cleaning fluid is sprayed by a spray device, and at least some of the suctioned cleaning fluid is suctioned out of the mouth via a suction port that is deployed on or within the spray device.

In some embodiments, the expanding is performed by inflating the fluid blocking element.

In some embodiments, the fluid blocking element is compressible and outwardly biased so that it is inserted into the mouth when compressed, and expands upon insertion into the mouth.

In some embodiments, the blocking element is inserted when the tube is within the mouth of the human subject so that the midsection channel of the blocking element fits snugly and reversibly around an outer surface of the tube.

In some embodiments, the fluid blocking element is reversibly inflatable to simultaneously press against the soft palate and the tongue.

It is now disclosed a system for oral care of patients ventilated with an endotracheal tube (ETT), the system comprising: a. a deformable fluid blocking element reversibly mountable around an ETT; and b. a suction tube attached to the fluid blocking element so that a distal end of the suction tube is situated and oriented to suck fluids from a proximal surface of the fluid blocking element, the fluid blocking element having: i. a midsection channel sized to fit snugly and reversibly around the ETT when the fluid blocking element is mounted thereto; and ii. an outer surface configured, when the fluid blocking element is mounted, to fit snugly and reversibly into the back of the mouth of an adult human patient so as to span both lateral left-to-right tonsils and tongue-soft palate dimensions thereof, wherein (A) the fluid blocking element is reversibly inflatable to simultaneously press against the soft palate and the tongue and/or (B) the fluid blocking element is collapsible and outwardly biased so that when placed in the back of the human mouth, the fluid blocking element expands so as to simultaneously press against the soft palate and the tongue.

It is now disclosed an oral cleaning method comprising: a. inserting a deformable fluid blocking element into a mouth of an adult human subject who is ventilated with an endotracheal tube (ETT), the fluid blocking element having a midsection channel sized to fit snugly and reversibly around the ETT, the fluid blocking element being inserted so the ETT passes through the midsection channel; and b. concurrent with or subsequent to the insertion, expanding the deformable fluid blocking element so that an outer surface thereof fits snugly and reversibly into the back of the mouth of the adult human subject so as to span both lateral and tongue-soft palate dimensions thereof.

It is now disclosed a system for oral care of patients ventilated with an endotracheal tube (ETT), the system comprising: a deformable fluid blocking element reversibly mountable around the ETT, the fluid blocking element having: (i) a midsection channel sized to fit snugly and reversibly around the ETT when the fluid blocking element is mounted thereto; and (ii) an outer surface configured, when the fluid blocking element is mounted, to fit snugly and reversibly into the back of the mouth of an adult human patient so as to span both lateral left-to-right tonsils and tongue-soft palate dimensions thereof, wherein the fluid blocking element is configured so that when mounted to the ETT, (A) the fluid blocking element is reversibly inflatable to simultaneously press against the soft palate and the tongue and/or (B) the fluid blocking element is collapsible and outwardly biased so that when placed in the back of the human mouth, the fluid blocking element expands so as to simultaneously press against the soft palate and the tongue.

In some embodiments, the deformable fluid blocking element is side-mountable to the ETT.

In some embodiments, further comprising a suction tube attached to the fluid blocking element so that a distal end of the suction tube is oriented to suck fluids from a proximal surface of the fluid blocking element.

In some embodiments, further comprising: a spray-head device having at least one inlet for receiving pressurized stream of fluid and at least one fluid-delivery orifice for liquid-gas mixture spray delivery of into the mouth at a time when the fluid blocking element is deployed therein.

In some embodiments, the spray-head device further includes a suction lumen and a suction orifice, both the fluid-delivery orifice and the suction orifice being disposed at or near a distal end of the spray-head device.

In some embodiments, the spray-head device is operative to deliver spray of a sufficient strength to remove dental plaque.

In some embodiments, the fluid blocking element comprises one or more of: a compressible foam, or a sponge, or an inflatable balloon, or a diaphragm.

In some embodiments, the channel is sized to fit over, or capable to stretch open over, an ETT of diameter of at least 7 mm or at least 8 mm or at least 10 mm.

In some embodiments, fluid blocking element dimensions span both lateral width of at least 3 cm and tongue-soft palate height of at least 1.5 cm.

In some embodiments, the ETT includes a Murphey eye at a distal end thereof.

### BRIEF DESCRIPTION OF THE DRAWINGS

FIG. 1A illustrates an example of an endotracheal tube (ETT) (PRIOR ART).

FIG. 1B illustrates various elements of the human oral cavity (PRIOR ART).

FIG. 1C illustrates the oral cavity of FIG. 1B when intubated with an ETT (PRIOR ART).

FIGS. 2-4 illustrate systems, apparatus and portions thereof of a fluid blocking element part of kit for oral cleaning according to some embodiments of the invention.

FIGS. 5-8 illustrate systems, apparatus and portions thereof of a spray cleaning element part of kit for oral cleaning according to some embodiments of the invention.

FIG. 9 illustrates a subject intubated with an ETT where an expandable fluid-blocking element is mounted (e.g. reversibly mounted) thereon at a location proximal to a permanently-attached cuff.

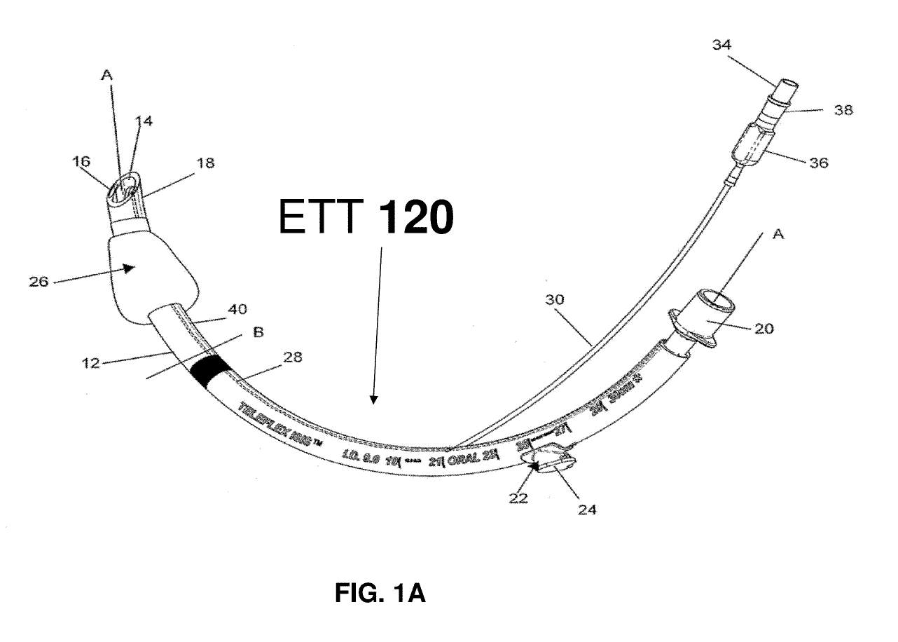

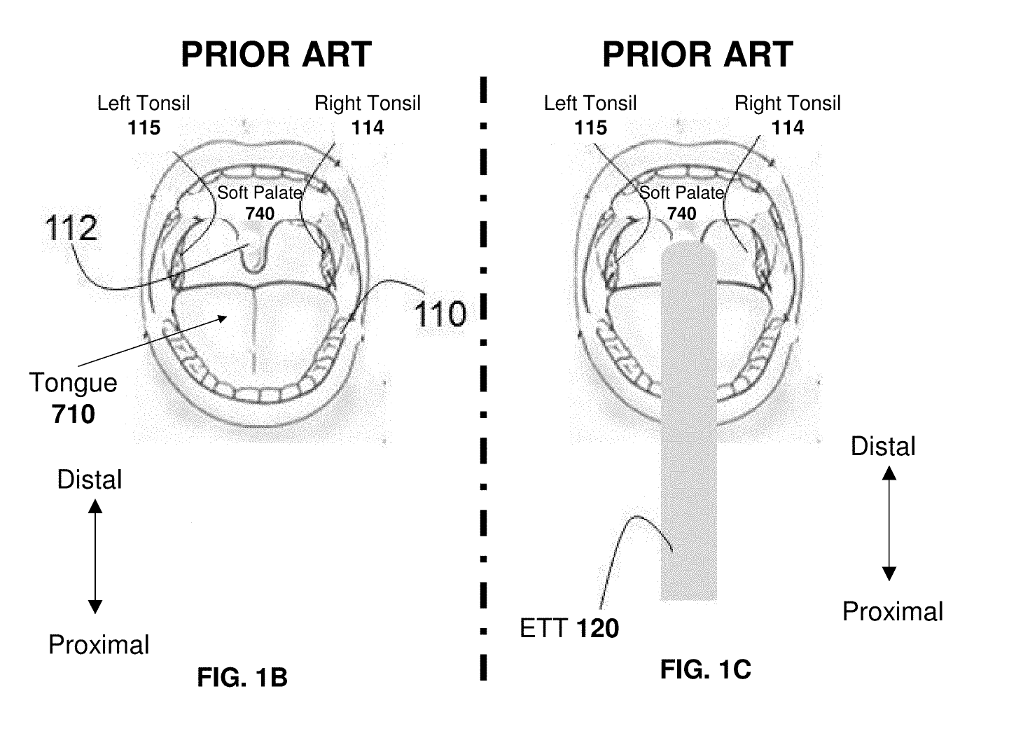

### DETAILED DESCRIPTION OF EMBODIMENTS

Embodiments of the present invention relate to systems, methods and kits for cleaning the oral cavity of an intubated subject.

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner —including any combination of features—any combination of features can be included in any embodiment and/or omitted from any embodiments.

In relation to the figures the following numbers indicate:
110—back teeth
112—uvula
114—right tonsil
115—left tonsil
200—fluid blocking element
205—mid-section channel of fluid blocking element 200.
210—ETT-facing surface of channel in blocking element
220—main suction tube
221—right suction branch
222—right suction inlet
223—left suction branch
224—left suction inlet
225—suction tube end connector
226—suction branches (221 and/or 223) linkage into the ETT 220
230—optional inflation tube
335—inflation tube end connector
500—spray device
510—elongated body of spray device
520—spray outlet orifice
521—liquid lumen
523—gas lumen
525—mixing chamber
526—acceleration duct
527—distal outlet of acceleration duct
531—liquid delivery tube
532—liquid tube distal connector
533—gas delivery tube
534—gas tube distal connector
541—liquid reservoir
543—pressurized gas source
560—elevation bump
585—protective shield
590—spray
620,630,640,650—various schematic surfaces of a fluid-blocking element 200
710—tongue of human subject
740—soft palate of human subject

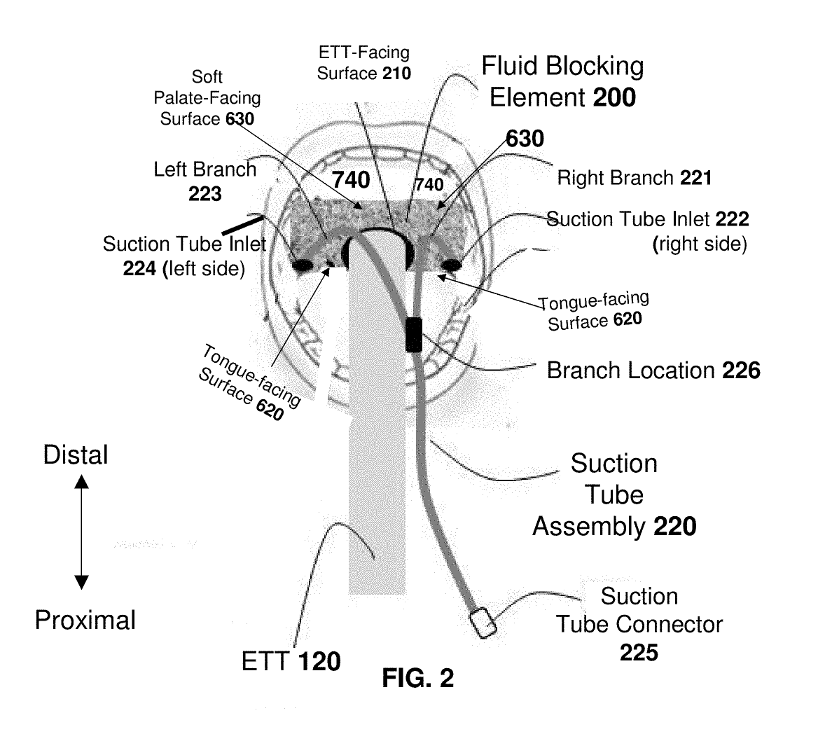

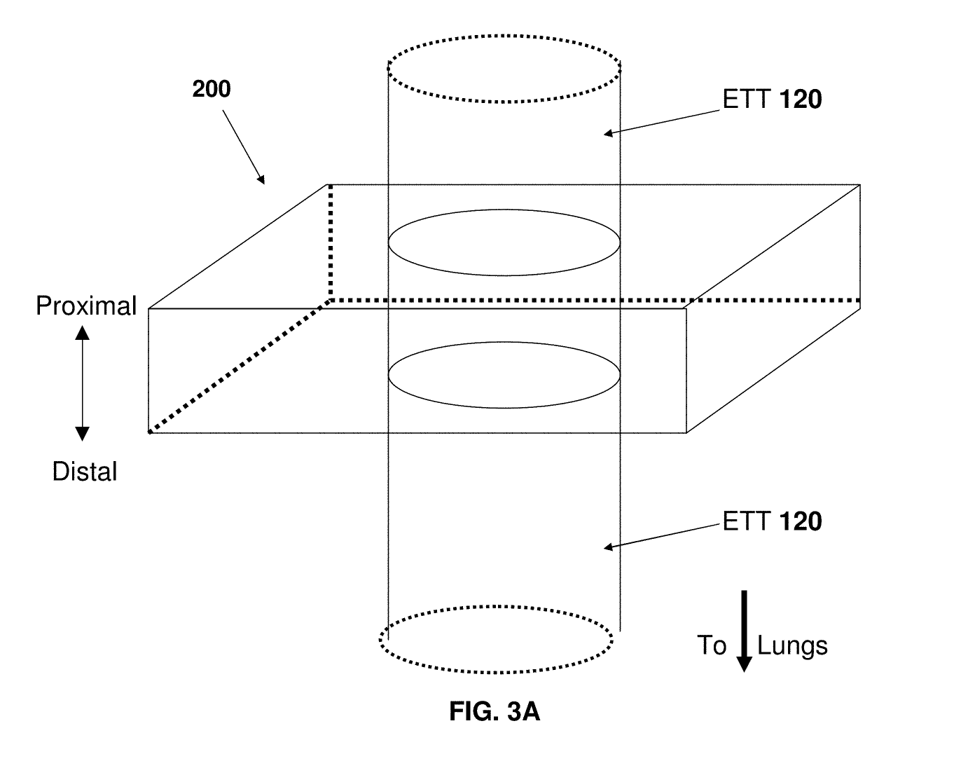

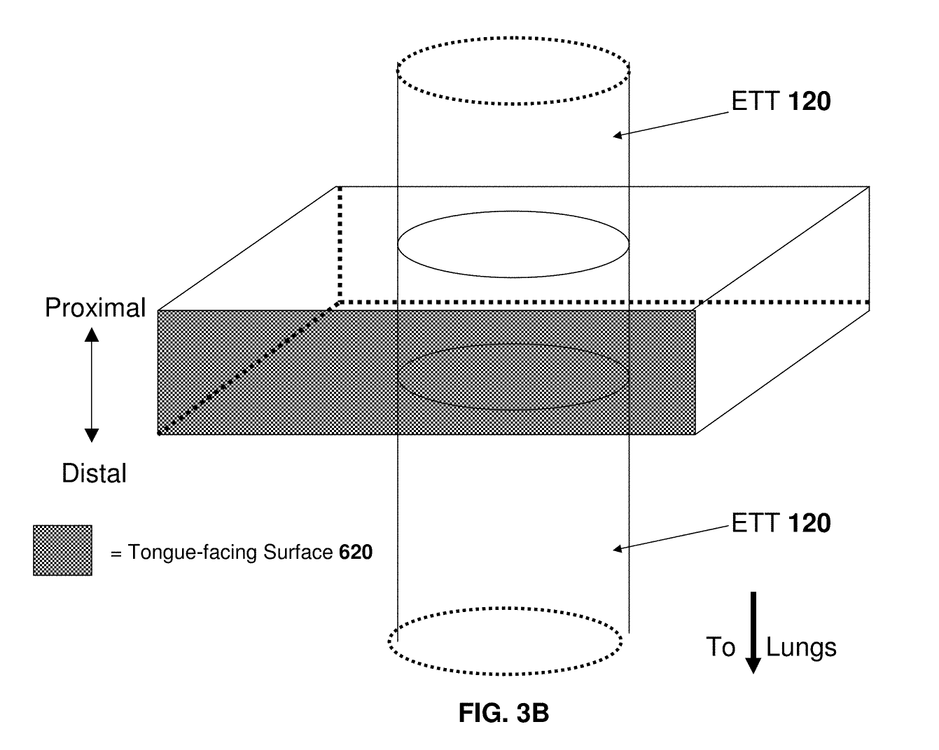

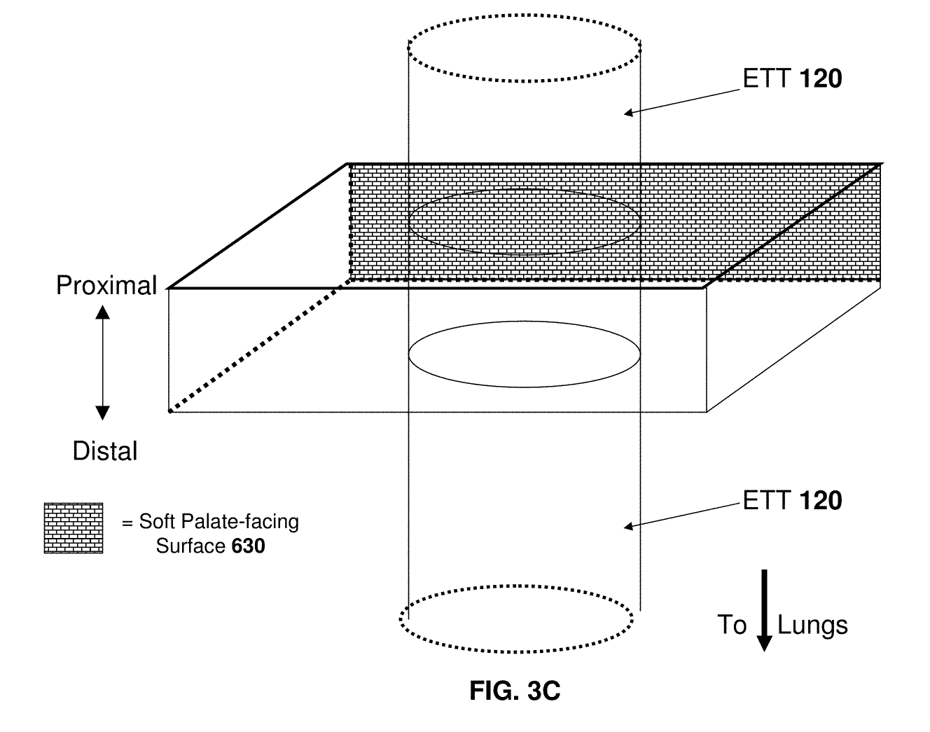

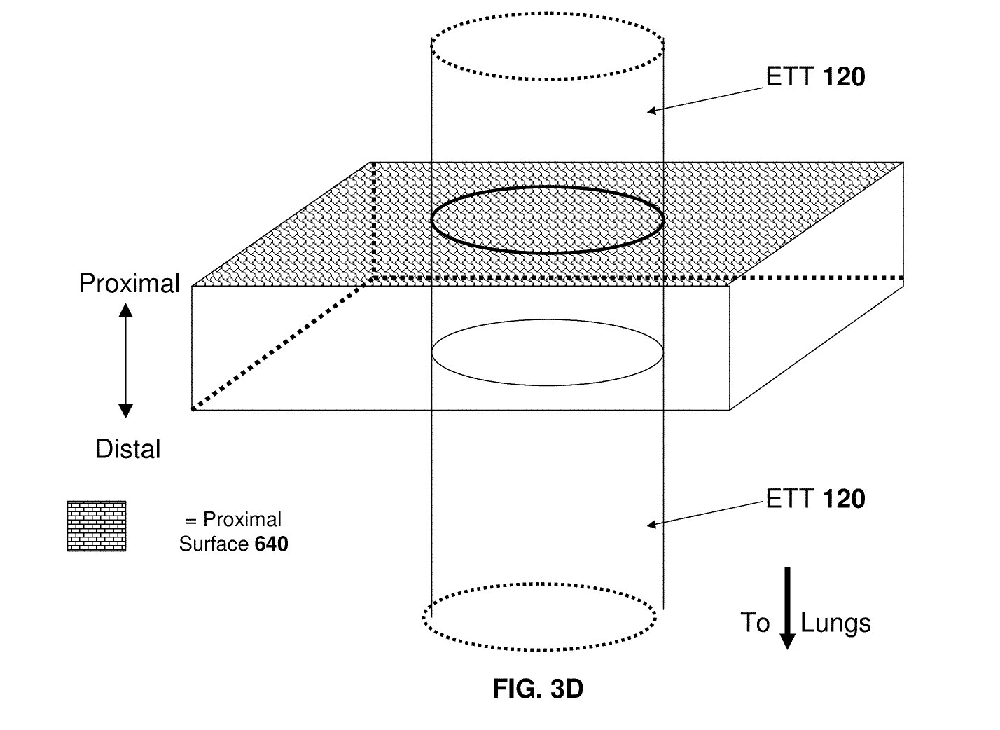

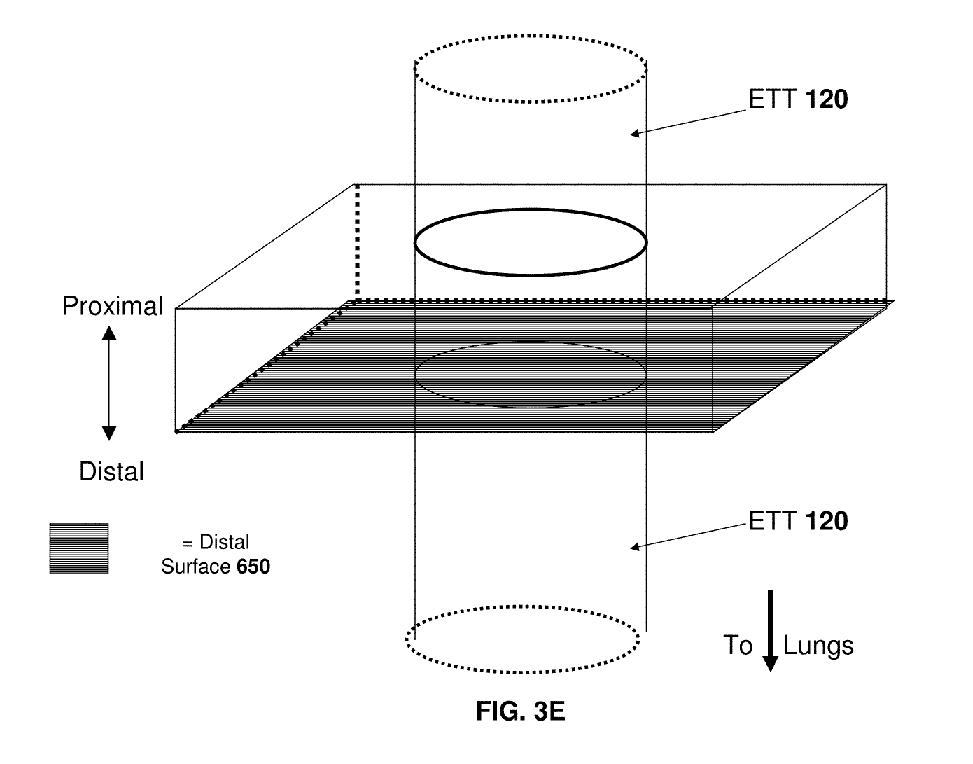

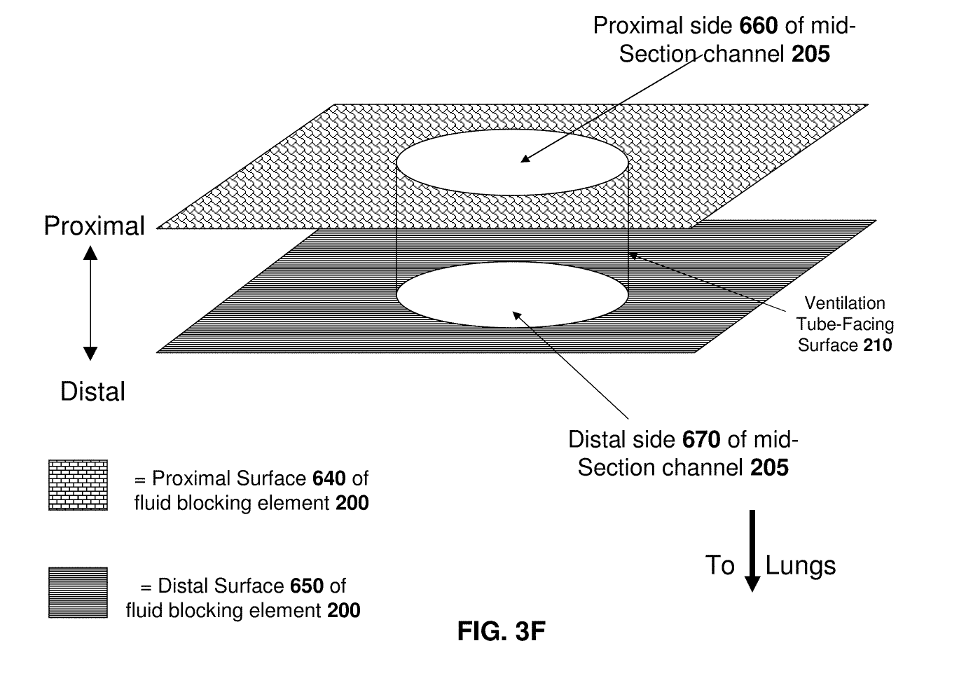

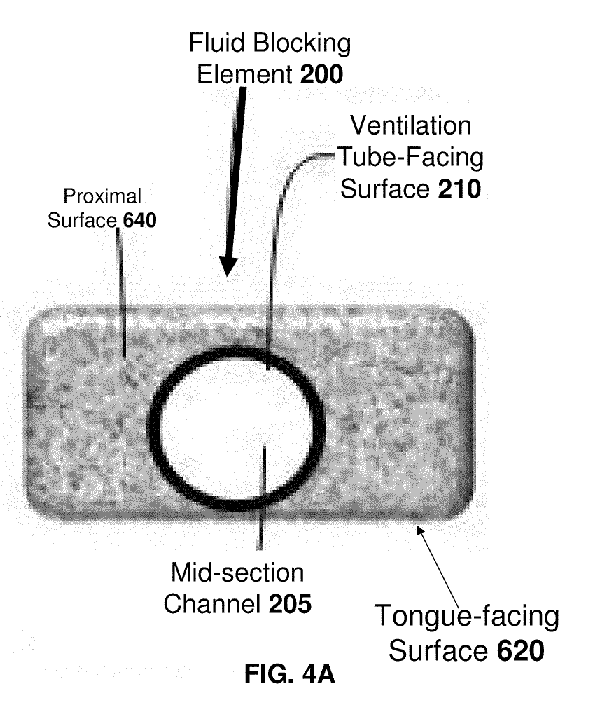

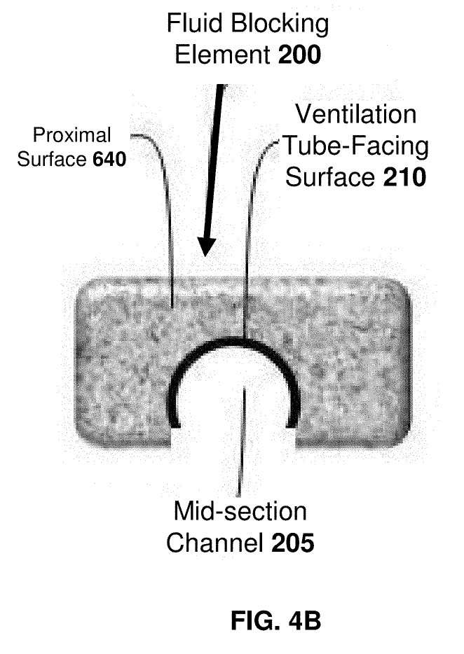

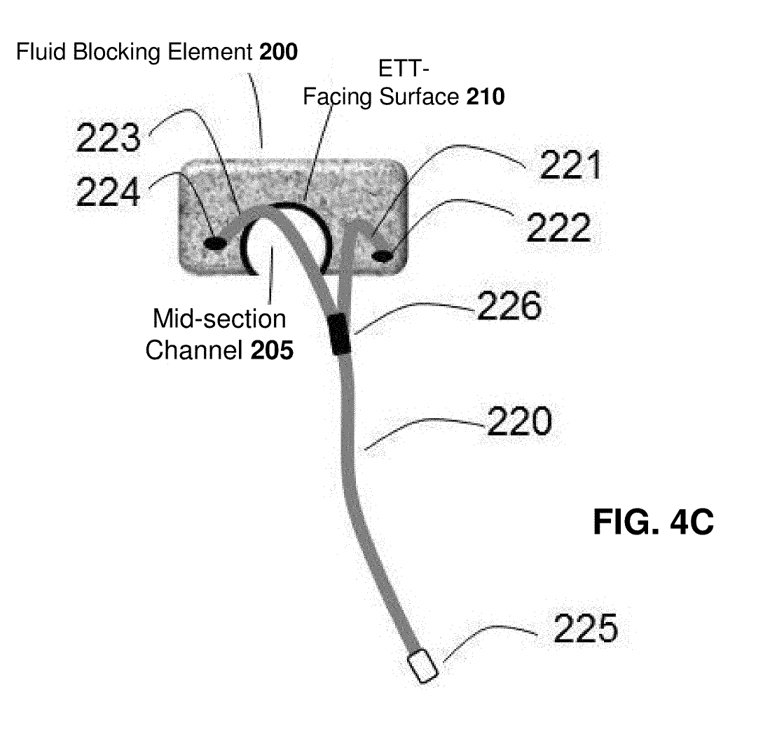

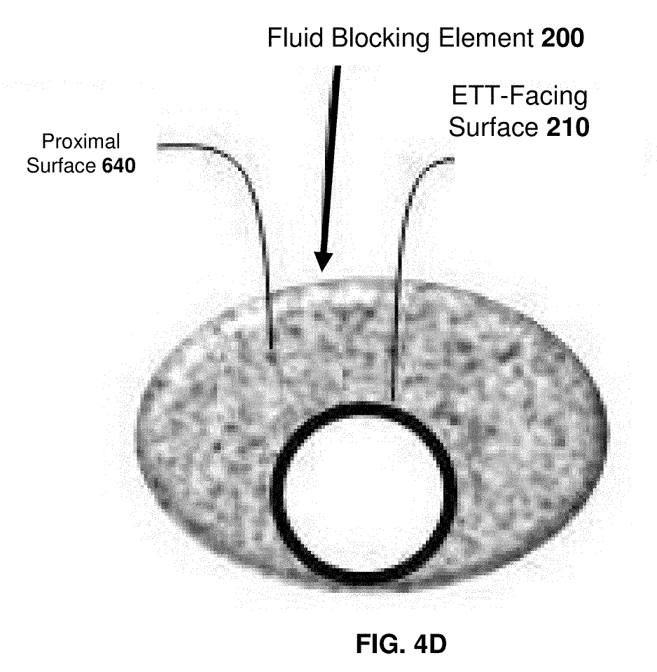

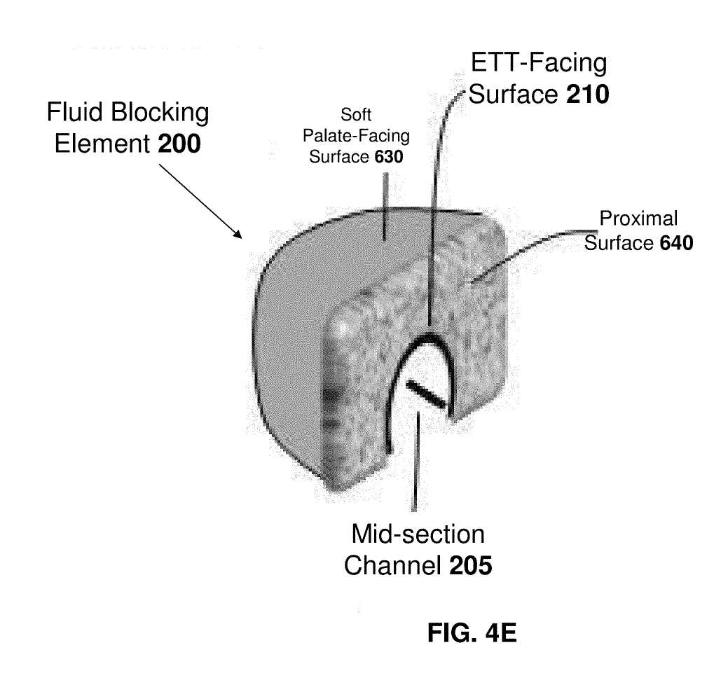

In the non-limiting example of FIG. 2, the system includes: a fluid blocking element 200 having a channel (see element 205 of FIGS. 3F and 4A-4E) through which ETT 120 passes. The fluid blocking element 200 fits snugly into the back of the subject's mouth so as to substantially span both ‘width’ dimensions of the mouth—i.e. the lateral dimension between the left 115 and right 114 tonsils and the ‘soft palate-tongue’ dimension between the subject's soft palate 740 and the tongue 710. Fluid blocking element 200 is dimensioned so as to substantially fill an entirety of the annular region outside of ETT 120 within the back of the subject's mouth.

In order to clean the subject's gums and/or teeth, is possible to deliver a pressurized fluid (e.g. a spray or mist or jet of fluid) into the subject's mouth at a time when the fluid blocking element 200 is deployed within the back of the subject's mouth so as to substantially fill the entirety of the annular region outside of ETT 120 within the back of the subject's mouth. The presence of fluid-blocking element substantially blocks fluids associated with the gums/teeth cleaning process (i.e. ‘cleaning fluids’) from penetrating deeper into the patient's oral cavity. Because the cleaning fluids may include oral-cavity microbes (e.g. derived from plaque or oral biofilm which may mix with the cleaning fluids after introduction into the oral cavity), blocking of these microbes-containing fluids by fluid blocking element 200 may reduce or eliminate collateral contamination of the trachea associated with the cleaning of the teeth or gums.

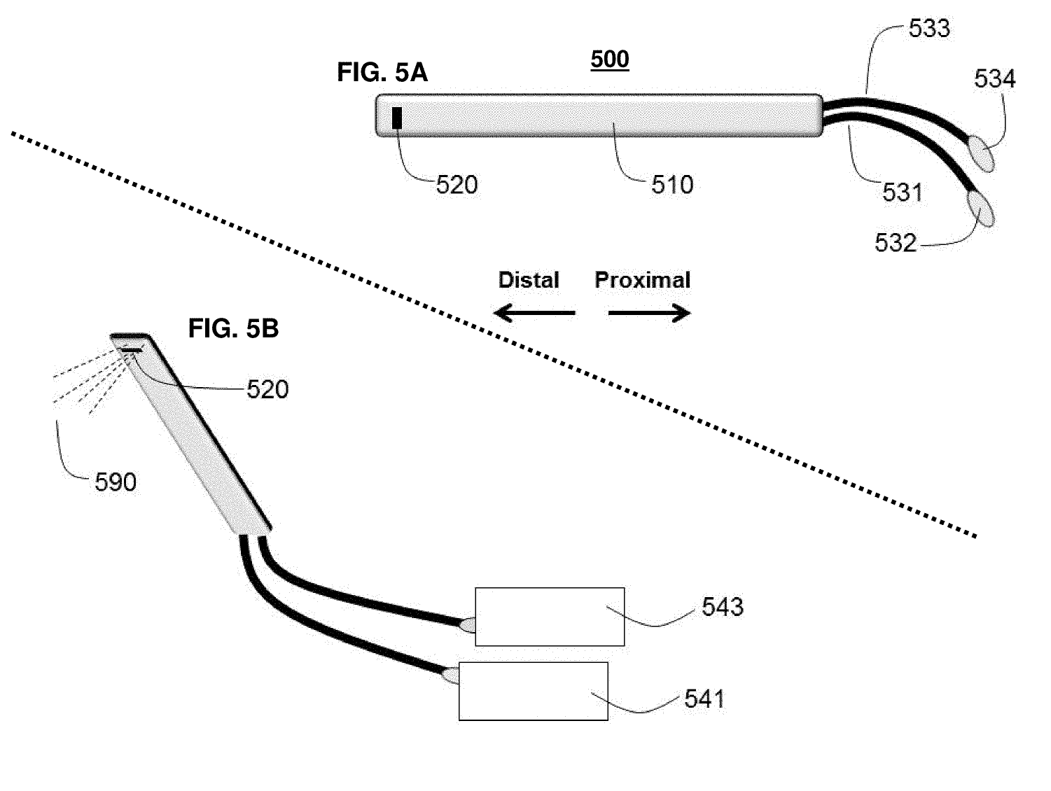

Some embodiments relate to kits including both the fluid-blocking element 200 and a spray-head device for delivering a pressurized stream of fluid into the subject's mouth. Other embodiments relate to systems including fluid-blocking element 200 which may or may not include the spray-head device. A more detailed discussion about example spray-head devices with reference to FIGS. 5-7 is provided below.

In order to further protect the trachea from microbes mixed into the cleaning fluid, and in order to further reduce the rate at which contaminating cleaning fluid leaks into locations below fluid-blocking element 200, it is possible to suction the contaminated or ‘used’ cleaning fluids from a location above the fluid-blocking element 200. In the non-limiting example of FIG. 2, a tube assembly 220 of one or more tubes for suctioning away cleaning fluids is provided. In the particular example of FIG. 2, tube assembly 220 may include multiple branches 221, 223 each having its own respective port 222, 224. On the proximal end, a suction tube connector 225 may be connected to a source of negative pressure.

FIG. 2 illustrates a preferred embodiment of the blocking element 200 further comprising attached suction tube 220. To enable better suctioning action on both sides of an ETT, it is preferred to have both a right suction branch tube 221 to one side of the channel 205, and a left suction branch tube 223 on the other side of the channel 205, each of which connect at its proximal end to the main suction tube 220. The right suction branch 221 comprises a distal suction port 222, and the left suction branch 223 comprises a distal suction port 224. The proximal end of the suction tube 220 includes a connector 225 suitable to connect to a suction source.

In some embodiments, one or more tubes of tube assembly 220 is attached to the fluid-blocking element 200 so that a distal end thereof (e.g. 222 or 224 as illustrated in FIG. 2) is oriented to suck fluids from a proximal surface (see element 640 of FIG. 3D) of fluid-blocking element 200. For example, port 222 or 224 may be held on or slightly above proximal surface 640 of fluid-blocking element 200.

As noted above, fluid-blocking element 200 may snugly fit within the subject's mouth so as to substantially fill the entire annular region outside of the ETT 120 and within the back of the subject's mouth. Although fluid-blocking element 200 is not required to be shaped as a rectangular prism, roughly speaking, fluid-blocking element 200 may include six ‘outer’ surfaces: (i) a soft-palate facing surface 630 facing and in contact with (or nearly in contact with) soft palate 740; (ii) a tongue-facing surface 620 facing and in contact with (or nearly in contact with) tongue 710; (iv) left-tonsil and right-tonsil facing surfaces (not numbered in the figures); (v) proximate surface 640 and (vi) distal surface 650.

The term ‘outer surface’ of the fluid blocking element refers to the union of all of the six aforementioned surfaces. This is in contrast to the ‘inner surface’ discussed below which is the surface of the midsection channel 205 faces the ETT. In order for the ‘outer surface’ to fit snugly into the back of the mouth, the following two activities must occur simultaneously (i) the soft-palate facing surface 630 must press onto the soft palate and (ii) the tongue-facing surface 620 must press onto the tongue.

In some embodiments, the fluid blocking element permanently has an annular shape and is top-mounted onto the ETT (e.g. by sliding onto the ETT at the top/proximal end). In preferably embodiments, the fluid blocking element has a ‘clip on’ or is otherwise side-mountable to the ETT. This allows for mounting the fluid blocking element to the ETT and removing the fluid blocking therefrom to clean the patient's teeth with no need to even briefly interrupt ventilation of the patient.

Fluid-blocking element 200 may also include an inner surface—i.e. a surface of the midsection channel 205 through which ETT 120 passes. This inner surface is referred to as ETT-facing surface 210 and is typically shaped like a side of a cylinder or a portion thereof. When ETT 120 passes through the midsection channel 205, there is a snug fit between an outer surface of ETT 120 and ETT-facing surface 210.

FIG. 3A-3F illustrate these various surfaces 620, 630, 640, 650, 210 for the non-limiting case of a fluid-blocking element 200 shaped as a rectangular prism.

In the example of FIGS. 3A-3F, fluid-blocking element 200 (and proximal 640 and distal 650 surfaces thereof) is annular in shape. This is also the case for the example of FIGS. 4A and 4D. Although fluid-blocking element 200 is dimensioned to substantially (but not necessarily entirely fill) fill an entirety of the annular region outside of ETT 120 within the back of the subject's mouth, there is no requirement that fluid-blocking element 120 itself have an annular shape.

Also illustrated in FIG. 3F are proximal side 660 of mid-section channel 205 and distal side 670 of mid-section channel 205.

Alternatively, as illustrated in FIGS. 4B and 4E, fluid-blocking element 200 and mid-section channel 205 thereof need to surround an entirety of ETT 120. For the present disclosure, when the mid-section channel 205 is configured to fit snugly ‘around’ the ETT, it only needs to surround a majority of orientations of the ETT 120 in a ‘cross section plane’ of ETT perpendicular to an elongate axis thereof—for example, to define an arc of at least 180 degrees or at least 270 degrees. Thus, the term ‘around’ is not synonymous with ‘surround’ which would require an arc of 360 degrees.

In some embodiments, employing a fluid blocking element 200 that is not annular in shape allows for one to ‘clip on’ blocking element onto ETT 120 and ‘clip off’ blocking element from ETT 120 without an need to slide into place the fluid blocking element from an end (e.g. proximal end) of ETT 120. The “open arc” fluid channels 205 illustrated in FIGS. 4B and 4E may facilitate this process.

One advantage of the ‘open arc’ configuration is that the fluid blocking element can be side-mountable to the ETT. In another example, the fluid blocking element is sometimes annular in shape and sometimes has an open arc and is also side-mountable.

Fluid-blocking element 200 may be compressible and/or expandable to snugly fit within the human oral cavity, particularly at the back of the mouth behind the back teeth 110 and near the soft palate 740, spanning the width between the left 115 and right 114 tonsils. For that purpose, blocking element 200 may, in different embodiments, be primarily constructed from foam, and/or sponge, and/or diaphragm, and/or inflatable balloon.

In some embodiments, ETT-facing surface 210 of midsection channel 205 may be made of a relatively stiff non-compressible material. In such case, where the stiff surface 210 is an arc of more than 180 degrees, the stiff surface 210 can clip-on with stronger attachment on the ETT 120.

Because ETT 120 passes through blocking element 200 (i.e. through mid-section channel 205 thereof), a diameter of channel 205 is sized to enable an outer surface ETT 120 to go through blocking element 200. Typical outer diameters of ETT (e.g. ETT tubes) are in the range of 6 mm-13 mm.

As noted above, fluid blocking element 200 is not required to be shaped as a rectangular prism. FIG. 4E is a 3D view of blocking element 200 where a soft palate-facing surface 630 has a shape that may better match that of the soft-palate to allow a snug contact with soft-palate 740.

FIGS. 5-7 refer to non-limiting examples of spray-head devices. In FIGS. 5-7, the following elements are labeled: 500—spray device; 510—elongated body of spray device; 520—spray outlet orifice; 521—liquid lumen; 523—gas lumen; 525—mixing chamber; 526—acceleration duct; 527—distal outlet of acceleration duct; 531—liquid delivery tube; 532—liquid tube distal connector; 533—gas delivery tube; 534—gas tube distal connector; 541—liquid reservoir; 543—pressurized gas source; 560—elevation bump and 590—spray.

FIG. 5a and FIG. 5b illustrate an embodiment of the spray device 500 for teeth cleaning, comprising: and elongated body 510 of dimensions (length/width) similar to a tooth brush, spray outlet 520 near the distal end of the elongated body 510, liquid delivery tube 531, and gas delivery tube 533. The liquid and gas tubes are preferably connected at a proximal portion of the elongated body 510.

In embodiments, the liquid delivery tube 531 and gas delivery tube 533 are detachable/re-connectable from/to the elongated body 510. Thereby, the elongated body 510 may be disposable while the delivery tubes can be re-used with other main body 510 devices.

In operation, the spray device is to be connected with a pressurized gas source 543, and to a liquid reservoir 541. The liquid reservoir may or may not be pressurized relative to atmospheric pressure. In embodiments, the gas source is pressure is between 2-8 atm, in order to create spray 590 suitable for cleaning of dental biofilm and/or plaque.

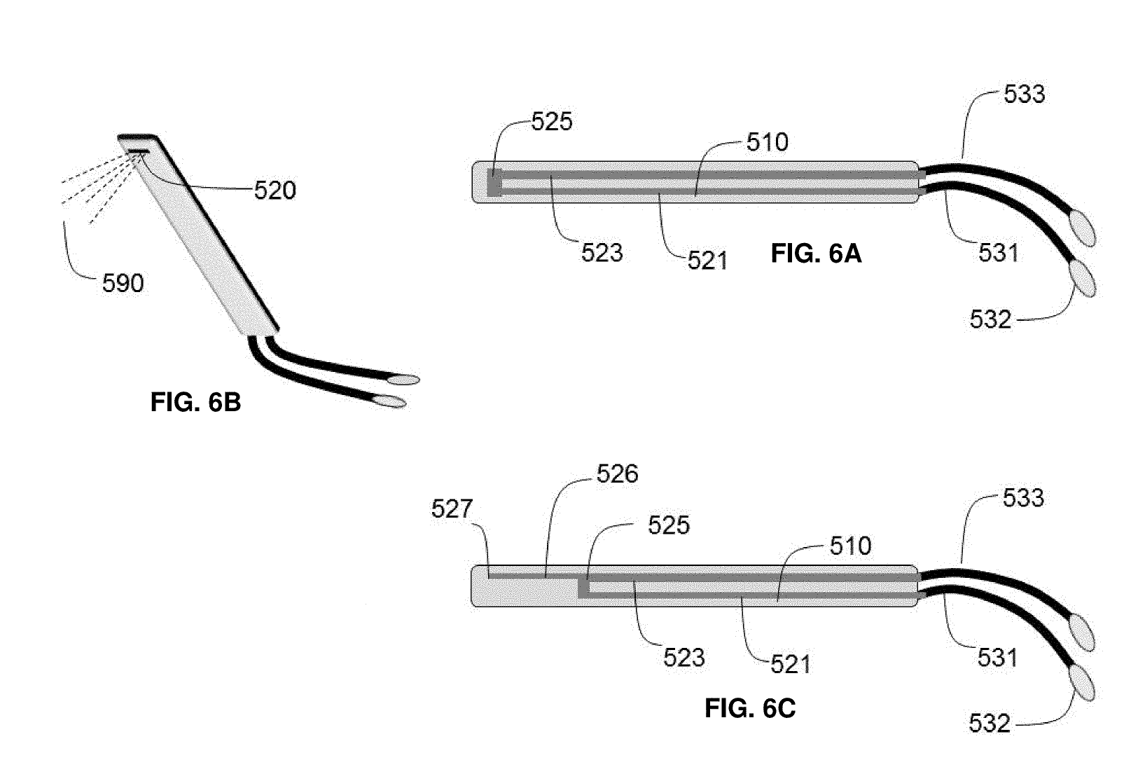

FIG. 6a illustrates embodiments of the spray device 500 of the type illustrated in FIG. 6b. In particular, lumen 521 is in fluid communication between the proximal inlet of the liquid delivery tube 531 and a distal mixing chamber 525, lumen 523 is in fluid communication between the proximal inlet of the gas delivery tube 533 and a distal mixing chamber 525. The mixing chamber 525 is in fluid communication with the spray outlet orifice 520.

FIG. 6c illustrates embodiments of the spray device 500 of the type illustrated in FIG. 4b, emphasizing the option of an acceleration duct 526 connecting and in fluid communication from the mixing chamber 525 to the spray outlet orifice 520. As illustrated in FIG. 6c, it should be understood that the mixing chamber 525 may consist simply of a tube junction of size comparable any of the lumens 521 or 523 diameters.

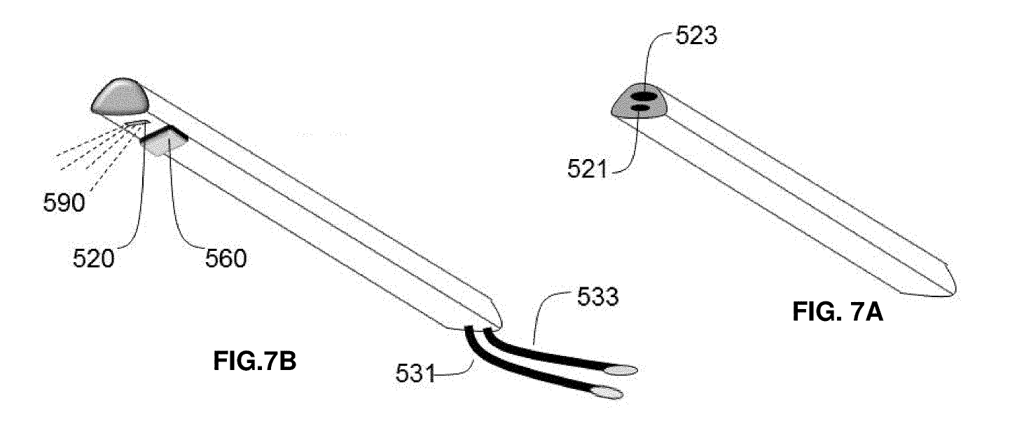

FIGS. 7a and 7b illustrate a similar embodiment of the spray device 500. The difference emphasized in the cross-section view 4c is that the lumen 523 is place behind lumen 521 (with respect to the facet of the spray outlet orifice 520).

In embodiments, the lumens 521 and 523 are simultaneously extruded.

In operation of the spray device 500, the gas streams (flows) intercept the liquid flow at an angle, preferably approximately perpendicularly. The velocity of the gas flows is sufficient to break up the liquid stream into a spray of droplets of desired size and velocity, optionally accelerating them through an acceleration duct and out of the orifice 520 at a desired high velocity effective for removal of biofilm and/or plaque from teeth and/or gums.

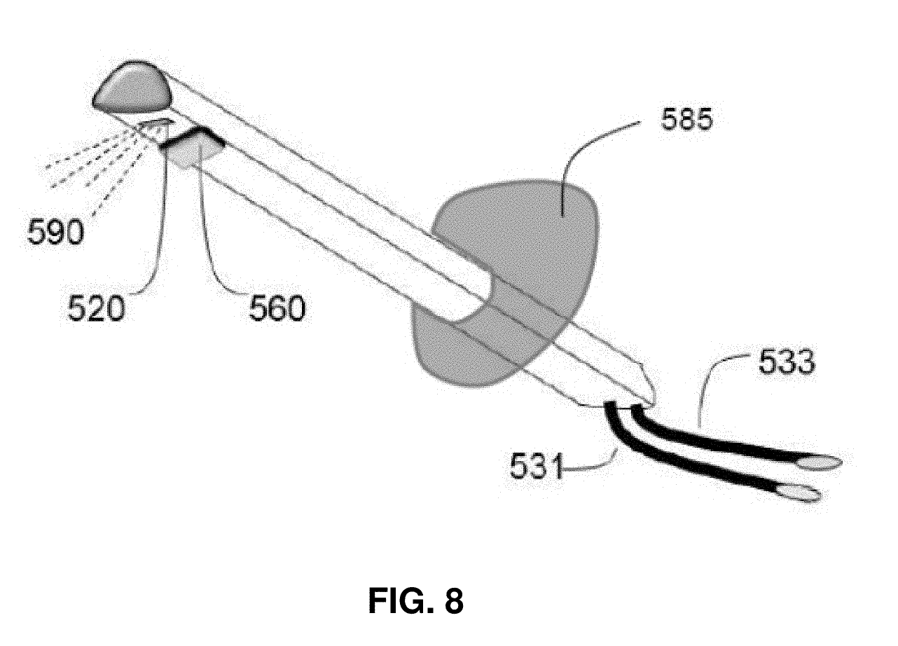

FIG. 8 illustrates a preferred embodiment where a protective shield 585, e.g., in the form of a flat plate, is mounted on the spray device main body 510. The shield 585 functions to block spray deflected from the mouth to reach the operator of the spray device (e.g., a nurse cleaning the mouth of ICU patient).

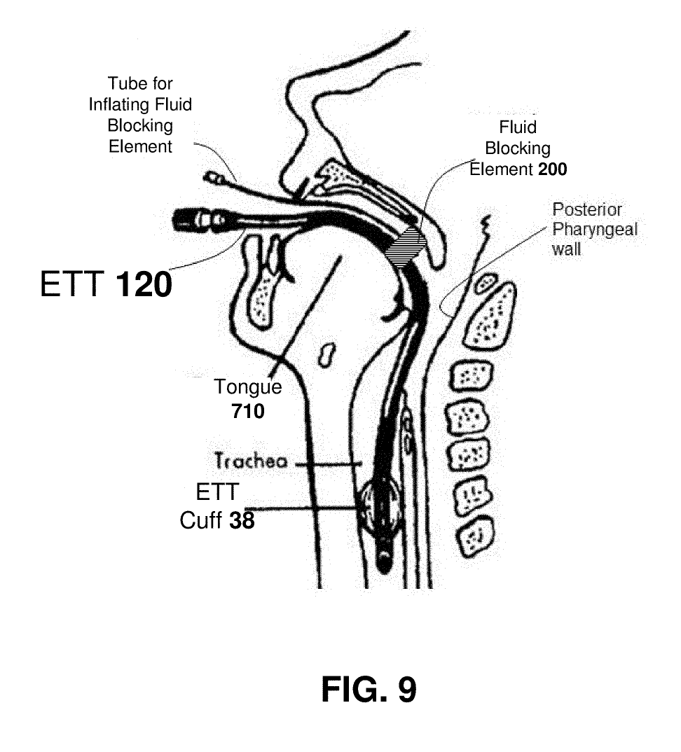

FIG. 9 illustrates the mounted fluid blocking element mounted to the ETT at a location a. above the epiglottis, and/or b. not touching the posterior pharyngeal wall.

### Definitions

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

Some embodiments relate to a ‘width’ of an objection—for example, a ‘width’ of an elongate flexible main body 210 or a width of an orifice(s) or a width of a lumen. A ‘width’ is defined as the square root of the cross section.

Additional Discussion

Embodiments of the present invention relate to a system for oral care of patients ventilated with an ETT, the system comprising a deformable fluid blocking element that is reversibly mountable around the ETT—e.g. slidable along the ETT when mounted thereon. The ‘reversibly mountable’ feature of the fluid blocking element means it can be mounted to and detached from the ETT, in contrast to any permanent attachment such as one based on gluing the fluid blocking element to the ETT.

The fluid blocking element has (i) a midsection channel 205 sized to fit snugly and reversibly around the ETT 120 when the fluid blocking element is mounted thereto; and (ii) an outer surface configured, when the fluid blocking element is mounted, to fit snugly and reversibly into the back of the mouth of an adult human patient so as to span both lateral left-to-right tonsils and tongue-soft palate dimensions thereof.

For the present disclosure, when the fluid blocking element fits “snugly” into the back of the mouth outer pressure is applied, by the fluid blocking element, simultaneously on both the tongue (e.g. the back of the tongue) and the soft palate. There is no requirement that outward pressure is applied upon any surface other than the on the tongue and the soft palate, although such pressure may, in fact, be applied.

In some embodiments, the fluid blocking element 200 is reversibly inflatable (e.g. including a balloon) to simultaneously press against the soft pallet and the tongue and/or the fluid blocking element 200 is collapsible and outwardly biased so that when placed in the back of the human mouth, the fluid blocking element expands so as to simultaneously press against the soft pallet and the tongue.

In exemplary embodiments, the fluid blocking element may be provided as a balloon and/or foam and/or sponge and/or diaphragm.

In some embodiments, the fluid blocking element permanently has an annular shape and is top-mounted onto the ETT (e.g. by sliding onto the ETT at the top/proximal end). Alternatively, the fluid blocking element has a ‘clip on’ or is otherwise side-mountable to the ETT. This allows for mounting the fluid blocking element to the ETT and removing the fluid blocking therefrom to clean the patient's teeth with no need to even briefly interrupt ventilation of the patient.

Some embodiments relate to an oral cleaning kit comprising: (i) the aforementioned reversibly mountable deformable fluid blocking element and (ii) a spray device for spraying a fluid into the oral cavity to clean the teeth. When fluid is sprayed into the intubated oral cavity, the presence of the fluid blocking element blocks fluids from penetrating into the airway below the blocking element.

In patients intubated with an endo tracheal tube (ETT), the ETT inhibits the swallowing ability. Hence cleaning the oral cavity with streams of liquid containing fluids is problematic, since oral biofilm and/or plaque contaminated fluids may descend into the trachea and the lungs. By combining the use of a fluids blocking element 200, the oral care kit facilitates safe use of fluids spray device 500 for cleaning of the teeth and/or gums of patients intubated with an endo tracheal tube (ETT).
Preferably, suctioning means are connected to the fluids blocking element 200, such that the contaminated cleaning fluids are not only blocked from descending into the trachea but are also suctioned out of the patient oral cavity.
In use of preferred embodiments, the fluids blocking element 200 is first fitted over the ETT at the back of the oral cavity of the intubated patient. Suctioning is operated to suction fluids proximally to the blocking element 200. The fluids spray device 500 is then operated to cleaning the patient oral cavity while suctioning.

It is now disclosed a system for oral care of patients ventilated with a ETT comprising: a deformable fluid blocking element reversibly mountable around a ETT, the fluid blocking element having a midsection channel sized to fit snugly and reversibly around the ETT when the fluid blocking element is mounted thereto; and an outer surface configured, when the fluid blocking element is mounted, to fit snugly and reversibly into the back of the mouth of an adult human patient so as to span both lateral left-to-right tonsils and tongue-soft palate dimensions thereof, wherein the fluid blocking element is reversibly inflatable to simultaneously press against the soft pallet and the tongue and/or the fluid blocking element is collapsible and outwardly biased so that when placed in the back of the human mouth, the fluid blocking element expands so as to simultaneously press against the soft pallet and the tongue.

In some embodiments, the deformable fluid blocking element is side-mountable to the ETT.

In some embodiments, the system further comprises a suction tube attached to the fluid blocking element so that a distal end of the suction tube is oriented to suck fluids from a proximal surface of the fluid blocking element.

In some embodiments, the system further comprises a spray-head device having at least one inlet for receiving pressurized stream of fluid and at least one fluid-delivery orifice for liquid-gas mixture spray delivery of into the mouth at a time when the fluid blocking element is deployed therein.

In some embodiments, the spray-head device further includes a suction lumen and a suction orifice, both the fluid-delivery orifice and the suction orifice being disposed at or near a distal end of the spray-head device.

In some embodiments, the spray is of sufficient strength to remove dental plaque.

In some embodiments, the fluid blocking element comprises one or more of a compressible foam, or a sponge, or an inflatable balloon, or a diaphragm.

It is now disclosed an oral cleaning method comprising: a. inserting, into a mouth of an adult human subject, a deformable fluid blocking element having a midsection channel sized to fit snugly and reversibly around a ETT having an outer diameter of at least 6 mm and at most 13 mm, b. concurrent with or subsequent to the insertion, expanding the deformable fluid blocking element so that an outer surface thereof fits snugly and reversibly into the back of the mouth of the adult human subject so as to span both lateral and tongue-soft palate dimensions thereof.

It is now disclosed an oral cleaning method comprising: a. inserting a deformable fluid blocking element 200 into a mouth of an adult human subject who is ventilated with a ETT, the fluid blocking element having a midsection channel 205 sized to fit snugly and reversibly around the ETT, the fluid blocking element being inserted so the ETT passes through the midsection channel 205;

b. concurrent with or subsequent to the insertion, expanding the deformable fluid blocking element so that an outer surface thereof fits snugly and reversibly into the back of the mouth of the adult human subject so as to span both lateral and tongue-soft palate dimensions thereof.

In some embodiments, the method further comprises spraying a cleaning fluid into the mouth of human subject so as to clean teeth thereof, wherein the fluid blocking element substantially blocks the cleaning fluid from penetrating into an airway of the human subject.

In some embodiments, the fluid comprises a gas-liquid mixture.

In some embodiments, the method further comprises suctioning away cleaning fluid which is retained in the mouth by the fluid blocking element.

In some embodiments, at least some of the suctioned cleaning fluid is suctioned out of the mouth via enters via a suction port deployed substantially on an upper surface of the fluid blocking element.

In some embodiments, the cleaning fluid is sprayed by a spray device, and at least some of the suctioned cleaning fluid is suctioned out of the mouth via a suction port that is deployed on or within the spray device.

In some embodiments, the expanding is performed by inflating the fluid blocking element.

In some embodiments, the fluid blocking element is compressible and outwardly biased so that it is inserted into the mouth when compressed, and expands upon insertion into the mouth.

In some embodiments, the blocking element is inserted when the tube is within the mouth of the human subject so that the midsection channel of the blocking element fits snugly and reversibly around an outer surface of the tube.

In some embodiments, the tube is a ETT.

In some embodiments, the tube is a tracheostomy tube.

In some embodiments, the fluid blocking element is reversibly inflatable to simultaneously press against the soft pallet and the tongue.

In some embodiments, the fluid blocking element is collapsible and outwardly biased so that when placed in the back of the human mouth, the fluid blocking element expands so as to simultaneously press against the soft pallet and the tongue.

Although various embodiments were describe with respect to assembled systems, it is appreciate that kits including one or more parts which, when assembled, form any presently disclosed apparatus or device or portion thereof are also in the scope of the invention. In different non-limiting examples, connector assembly 158 and/or input module assembly 156 and/or any port disclosed herein may be provided as parts which may be assembled on-site (e.g. in the intensive care ward). In some examples, sleeve 610 may be part of a kit and deployed on site. Other configurations are within the scope of embodiments of the invention.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

All references cited herein are incorporated by reference in their entirety. Citation of a reference does not constitute an admission that the reference is prior art.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to. The term “or” is used herein to mean, and is used interchangeably with, the term “and/or,” unless context clearly indicates otherwise. The term “such as” is used herein to mean, and is used interchangeably, with the phrase “such as but not limited to”.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

1. A reversibly/side mountable system for oral care of patients ventilated with an endotracheal tube (ETT), the system comprising:
a deformable fluid blocking element reversibly mountable and side-mountable around the ETT, the fluid blocking element having:
(i) a midsection channel sized to fit snugly and reversibly around the ETT when the fluid blocking element is mounted thereto; and
(ii) an outer surface configured, when the fluid blocking element is mounted, to fit snugly and reversibly into the back of the mouth of an adult human patient so as to span both lateral left-to-right tonsils and tongue-soft palate dimensions thereof,
wherein the fluid blocking element is configured so that when mounted to the ETT, (A) the fluid blocking element is reversibly inflatable to simultaneously press against the soft palate and the tongue and/or (B) the fluid blocking element is collapsible and outwardly biased so that when placed in the back of the human mouth, the fluid blocking element expands so as to simultaneously press against the soft palate and the tongue.

2-3. (canceled)

4. The system of claim 1 wherein the deformable fluid blocking element is configured so that when mounted to the ETT, the fluid blocking element does not fully surround the ETT with a permanent ring.

5. The system of claim 4 wherein the deformable fluid blocking element is configured so that when mounted to the ETT, the fluid blocking element fully surrounds the ETT with a reversibly openable ring.

6-10. (canceled)

11. A ventilation system comprising:
a. an endotracheal tube (ETT); and
b. a deformable fluid blocking element reversibly mounted around the ETT, the fluid blocking element having:
a midsection channel sized to fit snugly and reversibly around the ETT when the fluid blocking element is mounted thereto; and
an outer surface configured, when the fluid blocking element is mounted, to fit snugly and reversibly into the back of the mouth of an adult human patient so as to span both lateral left-to-right tonsils and tongue-soft palate dimensions thereof,
wherein (i) the fluid blocking element is reversibly inflatable to simultaneously press against the soft palate and the tongue and/or (ii) the fluid blocking element is collapsible and outwardly biased so that when placed in the back of the human mouth, the fluid blocking element expands so as to simultaneously press against the soft palate and the tongue.

12. The system of claim 11 wherein the fluid blocking element is reversibly mounted around a proximal half of the ETT.

13-24. (canceled)

25. A system for oral care of patients ventilated with an endotracheal tube (ETT), the system comprising:
a. a deformable fluid blocking element reversibly mountable around an ETT; and
b. a suction tube attached to the fluid blocking element so that a distal end of the suction tube is situated and oriented to suck fluids from a proximal surface of the fluid blocking element,
the fluid blocking element having:
i. a midsection channel sized to fit snugly and reversibly around the ETT when the fluid blocking element is mounted thereto; and
ii. an outer surface configured, when the fluid blocking element is mounted, to fit snugly and reversibly into the back of the mouth of an adult human patient so as to span both lateral left-to-right tonsils and tongue-soft palate dimensions thereof,
wherein (A) the fluid blocking element is reversibly inflatable to simultaneously press against the soft palate and the tongue and/or (B) the fluid blocking element is collapsible and outwardly biased so that when placed in the back of the human mouth, the fluid blocking element expands so as to simultaneously press against the soft palate and the tongue.

26-38. (canceled)
